(19) United States
(12) Patent Application Publication
(10) Pub. No.: US 2023/0293303 A1
(43) Pub. Date: Sep. 21, 2023

(54) ELECTRONIC PUMP ASSEMBLY FOR AN IMPLANTABLE DEVICE HAVING A FLOW MODIFIER

(71) Applicant: Boston Scientific Scimed, Inc., Maple Grove, MN (US)

(72) Inventors: Daragh Nolan, Via Youghal (IE); Barbara Belisa Soffiati, Clonmel (IE); Thomas Sinnott, Enniscorthy (IE); Evania Ann Mareena, Clonmel (IE); Shauna O'Meara, Carrick-On-Suir (IE); Eduardo Marcos Larangeira, Cork City (IE); Noel Smith, Windgap (IE); Daniel O'Malley, Tipperary (IE)

(21) Appl. No.: 18/183,708

(22) Filed: Mar. 14, 2023

Related U.S. Application Data
(60) Provisional application No. 63/269,446, filed on Mar. 16, 2022.

(51) Int. Cl. A61F 2/26; A61F 2/48

(52) U.S. Cl. CPC A61F 2/26; A61F 2/482; A61F 2/484; A61F 2250/0013; A61F 2250/0002

(57) ABSTRACT

According to an aspect, an electronic pump assembly includes a pump configured to move fluid between from or to a fluid reservoir of a device. The pump includes a passive valve. The electronic pump assembly includes a controller configured to actuate the pump and a flow modifier configured to restrict a flow of fluid entering or within the pump.

### CROSS-REFERENCE TO RELATED APPLICATION

This application claims priority to U.S. Provisional Patent Application No. 63/269,446, filed on Mar. 16, 2022, entitled “AN ELECTRONIC PUMP ASSEMBLY FOR AN IMPLANTABLE DEVICE HAVING A FLOW MODIFIER”, the disclosure of which is incorporated by reference herein in its entirety.

### TECHNICAL FIELD

This disclosure relates generally to bodily implants and more specifically to bodily implants, such as electronic implantable devices having an electronic-based pump and a flow modifier to increase an efficiency of the electronic-based pump.

### BACKGROUND

An implantable device may include an electronic-based pump to transfer fluid components of the implantable device. However, in some conventional electronic-based pump designs, there may be a pumping inefficiency for transferring fluid between the components of the implantable device.

### SUMMARY

According to an aspect, an electronic pump assembly includes a pump configured to move fluid between from or to a fluid reservoir of a device. The pump includes a passive valve. The electronic pump assembly includes a controller configured to actuate the pump and a flow modifier configured to restrict a flow of fluid entering or within the pump.

The electronic pump assembly may include one or more of the following features (or any combination thereof). The flow modifier includes a filter disposed within a fluid passageway connected to the pump. The filter is a first filter connected to an input of the pump, the electronic pump assembly including a second filter connected to an output of the pump. The filter includes a tubular member defining a cavity, the filter includes a filter component disposed within the cavity of the tubular member, the component defining a plurality of holes. The plurality of holes include linear holes. The plurality of holes include conical holes. The filter component includes a cylindrical portion having a first end portion and a second end portion, the first end portion including a flat surface, the second end portion including a conical portion. The flow modifier is defined by at least a portion of the passive valve. The pump includes a piezo element, a diaphragm actuator, and a base plate defining an inlet port and an outlet port. The pump includes a first layer of material defining a first passive valve and a second layer of material defining a second passive valve, the first and second layers of material being disposed between the diaphragm actuator and the base plate.

According to an aspect, an implantable device includes a fluid reservoir configured to hold fluid, an inflatable member, and an electronic pump assembly configured to transfer the fluid between the fluid reservoir and the inflatable member. The electronic pump assembly includes a pump configured to move fluid between from or to a fluid reservoir of a device, the pump including a first passive valve and a second passive valve, a controller configured to actuate the pump, and a flow modifier configured to restrict a flow of fluid entering or within the pump.

According to some aspects, the implantable device may include one or more of the following features. The flow modifier includes a filter disposed within a fluid passageway connected to the pump. The filter includes a tubular member defining a cavity, the filter includes a cylindrical member disposed within the cavity of the tubular member, the cylindrical member defining a plurality of holes. The plurality of holes include circular holes, Y-shaped holes, hexagonal holes, honeycomb holes, or holes arranged in a Gyroid lattice. The flow modifier is defined by at least a portion of the passive valve. The pump includes a piezo element, a diaphragm actuator, and a base plate defining an inlet port and an outlet port. The pump includes a first layer of material defining the first passive valve and a second layer of material defining the second passive valve, the first and second layers of material being disposed between the diaphragm actuator and the base plate. At least one of the first layer of material or the second layer of material is stiffer than a material of the diaphragm actuator.

According to an aspect, a method of controlling an implantable device includes detecting a signal to actuate a pump of an electronic pump assembly, the pump including a piezoelectric pump having a passive valve, transferring, by the pump, fluid from a fluid reservoir, and restricting, by a flow modifier, a flow of fluid entering the pump. In some examples, the flow modifier is disposed in a fluid passageway between the fluid reservoir and the pump.

### BRIEF DESCRIPTION OF THE DRAWINGS

FIG. 1A illustrates a device with a pump and a flow modifier according to an aspect.

FIG. 1B illustrates an example of the flow modifier according to an aspect.

FIG. 2A illustrates an example of the pump according to an aspect.

FIG. 2B illustrates an example of the pump with an inlet check valve in an open position and an outlet check valve in a closed position according to an aspect.

FIG. 2C illustrates an example of the pump with an inlet check valve in a closed position and an outlet check valve in a closed position according to an aspect.

FIG. 2D illustrates an example of the pump with an inlet check in a closed position and an outlet check valve in an open position according to an aspect.

FIG. 3 depicts a pressure graph without a flow modifier according to an aspect.

FIG. 4 depicts a pressure graph with a flow modifier according to an aspect.

FIG. 5A illustrates an inflatable device with a filter in a fluid passageway between a pump and a fluid reservoir according to an aspect.

FIG. 5B illustrates an example of the inflatable device according to an aspect.

FIG. 6 illustrates an inflatable device according to another aspect.

FIG. 7 illustrates an example of a filter according to an aspect.

FIG. 8 illustrates a filter component defining a plurality of holes according to an aspect.

FIG. 9 illustrates a filter component defining a plurality of holes according to an aspect.

FIG. 10A illustrates an example of a filter according to an aspect.

FIG. 10B illustrates an example of a filter component defining a plurality of holes according to an aspect.

FIG. 11A illustrates an example of a filter according to an aspect.

FIG. 11B illustrates an example of a filter component defining a plurality of holes according to an aspect.

FIG. 12A illustrates an example of a filter component defining a plurality of holes according to an aspect.

FIG. 12B illustrates a perspective of the filter component according to an aspect.

FIG. 12C illustrates another perspective of the filter component according to an aspect.

FIG. 12D illustrates another perspective of the filter component according to an aspect.

FIG. 12E illustrates another perspective of the filter component according to an aspect.

FIG. 13 illustrates an example of a filter according to an aspect.

FIG. 14A illustrates an example of a hole of the filter according to an aspect.

FIG. 14B illustrates another perspective of a filter component according to an aspect.

FIG. 14C illustrates another perspective of the filter component according to an aspect.

FIG. 14D illustrates another perspective of the filter component according to an aspect.

FIG. 15 illustrates an example of an inflatable penile prosthesis according to an aspect.

FIG. 16 illustrates an example of an artificial urinary sphincter device according to an aspect.

FIG. 17 illustrates a flowchart depicting example operations of controlling an implantable device according to an aspect.

### DETAILED DESCRIPTION

This disclosure relates to an electronic pump assembly of a device (e.g., an implantable device). The electronic pump assembly includes one or more pumps (e.g., electronic-based pump(s)) and a flow modifier that can increase the pumping efficiency of the pump(s). In some examples, a pump includes one or more passive valves, and the flow modifier is configured to increase the efficiency in the opening and closing of the passive valve(s), thereby increasing the pumping efficiency of the pump. In some examples, a loss of efficiency of a pump may exist due to the amount of time (e.g., longer time) to open and close the passive valve(s) and/or due to vibration effects in the fluid system. However, the flow modifier is configured to increase the pump efficiency by decreasing the amount of time to open and close the passive valve(s) and reduce the effect of vibrations on the pumping mechanism.

In some examples, the device is an artificial urinary sphincter device. In some examples, the device is an inflatable penile prosthesis. An implantable device may include an inflatable member, an electronic pump assembly, and a fluid reservoir. The electronic pump assembly may transfer fluid between the fluid reservoir and the inflatable member without the user manually operating a pump bulb. For example, the transfer of fluid between the inflatable member and the fluid reservoir is electrically controlled. In some examples, the implantable device is a drug pump such as an insulin or pain management drug pump that includes a piezoelectric (or other type of pump).

In some examples, the pump includes a piezoelectric diaphragm pump, which may include a piezoelectric element, a diaphragm actuator coupled to the piezoelectric element, and a base plate defining an inlet port and an outlet port. The pump includes one or more passive valves (e.g., check valves), which may include one or more layers of material that define ring member(s) (e.g., an O-ring). In some examples, the layer(s) of material that define the ring member(s) may be disposed between the diaphragm actuator and the base plate. On closure of the passive valve, the flow path is impeded by the interface between the diaphragm actuator and the ring member, which closes the fluid path. When the diaphragm actuator is released from the ring member, the fluid can flow.

In some examples, the flow modifier includes a filter disposed in a fluid passageway connected to the pump. The filter is configured to restrict the flow of fluid entering the pump. In some examples, the filter is disposed in a fluid passageway between a fluid reservoir and the pump. In some examples, the electronic pump assembly includes multiple filters, e.g., a first filter disposed between the fluid reservoir and the pump (e.g., connected to an input of the pump) and a second filter disposed between the pump and an inflatable member (e.g., connected to an output of the pump). In some examples, the filter includes a tubular member and a filter member (e.g., disc or cylindrical member) disposed within the tubular member, where the filter member defines a plurality of holes. In some examples, the filter member (e.g., disc or cylindrical member) includes a first end portion and a second end portion. In some examples, the first end portion includes a flat portion (e.g., flat surface). In some examples, the second end portion includes a conical portion (e.g., conical surface). In some examples, the holes of the disc member include linear holes. In some examples, the holes of the disc member include conical holes. In some examples, the holes of the disc member include Y-shaped holes, honeycomb holes, hexagonal holes, or holes arranged in a Gyroid lattice. In some examples, the sum of the area of the holes is substantially the same as the area of the filter member.

In some examples, the flow modifier is defined by one or more materials of the passive valve. In some examples, the material(s) of the passive valve may have different material properties or dimensions to enable a stiffer valve. For example, the material(s) of the passive valve may be constructed from a material that includes a higher stiffness than other materials of the pump (e.g., the material of a diaphragm actuator). In some examples, the material(s) of the passive valve may have a thickness greater than the thickness of other materials of the pump (e.g., the material of a diaphragm actuator). In some examples, the passive valve(s) include helical valves and the diameter(s) of the helical valves may be different (e.g., different from each other) and/or non-symmetrical to change the flow rate.

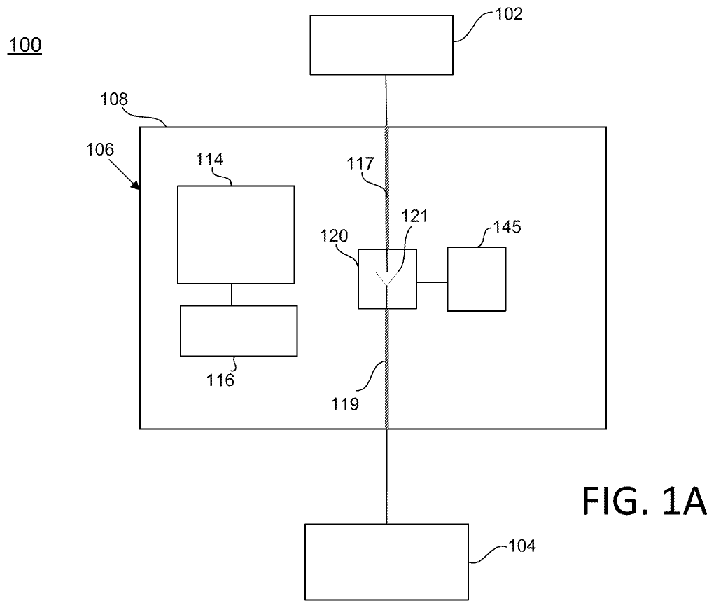

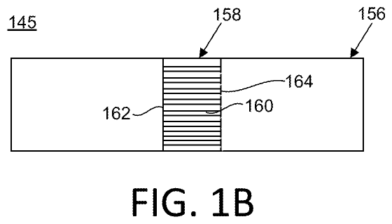

FIGS. 1A and 1B illustrate a device 100 with an electronic pump assembly 106 according to an aspect. In some examples, the device 100 is a medical device. In some examples, the device 100 is an implantable device such as an artificial urinary sphincter device, an inflatable penile prosthesis, or a drug pump (e.g., an insulin or pain management drug pump). In some examples, the device 100 is not implanted into the body of a patient. The electronic pump assembly 106 is configured to transfer fluid from a fluid reservoir 102. In some examples, the fluid is a liquid fluid such as a saline fluid. In some examples, the fluid is a gas (e.g., a gas under pressure). In some examples, the electronic pump assembly 106 is configured to transfer fluid between the fluid reservoir 102 and an inflatable member 104 (e.g., inflatable cuff, inflatable cylinder(s)). In some examples, the electronic pump assembly 106 does not include an inflatable member, where the electronic pump assembly 106 is configured to transfer fluid from the fluid reservoir to a body portion of the patient.

The electronic pump assembly 106 includes a controller 114, a battery 116, a pump 120 with a passive valve 121, and a flow modifier 145 that can increase the pumping efficiency of the pump 120. The flow modifier 145 is configured to increase the efficiency in the opening and closing of the passive valve 121, thereby increasing the pumping efficiency of the pump 120. In some examples, a loss of efficiency of a pump (e.g., an electronic-based pump) may exist due to the amount of time (e.g., longer time) to open and close the passive valve 121 and/or due to vibration effects in the fluid system. However, the flow modifier 145 is configured to increase the pump efficiency by decreasing the amount of time to open and close the passive valve 121 and reduce the effect of vibrations on the pumping mechanism.

In some examples, the pump 120 includes a piezoelectric diaphragm pump, which may include a piezoelectric element, a diaphragm actuator coupled to the piezoelectric element, and a base plate defining an inlet port and an outlet port. The pump 120 includes one or more passive valves 121, which may include one or more layers of material that define ring member(s) (e.g., an 0-ring). In some examples, the layer(s) of material that define the ring member(s) may be disposed between the diaphragm actuator and the base plate. On closure of the passive valve 121, the flow path is impeded by the interface between the diaphragm actuator and the ring member, which closes the fluid path. When the diaphragm actuator is released from the ring member, the fluid can flow.

The pump 120 is an electronically-controlled pump. The pump 120 may be electronically-controlled by the controller 114. For example, the pump 120 may be connected to the controller 114 and may receive a signal to actuate the pump 120. A pump 120 may be unidirectional in which the pump 120 can transfer fluid from the fluid reservoir 102 to the inflatable member 104 (or from the inflatable member 104 to the fluid reservoir 102). In some examples, a pump 120 is bidirectional in which the pump 120 can transfer fluid from the fluid reservoir 102 to the inflatable member 104 and from the inflatable member 104 to the fluid reservoir 102.

In some examples, the pump 120 is an electromagnetic pump that moves the fluid between the fluid reservoir 102 and the inflatable member 104 using electromagnetism. With respect to an electromagnetic pump, a magnetic fluid is set at angles to the direction the fluid moves in, and a current is passed through it.

In some examples, the pump 120 is a piezoelectric pump. In some examples, a piezoelectric pump may be a diaphragm micropump that uses actuation of a diaphragm to drive a fluid. In some examples, a piezoelectric pump may include one or more piezo pumps (e.g., piezo elements), which may be implemented by a substrate layer (e.g., a single substrate layer) of high-voltage piezo elements or may be implemented by multiple substrate layers (e.g., stacked substrate layers) of low-voltage piezo elements. In some examples, the pump 120 includes a plurality of micro-pumps (e.g., piezoelectrically-driven micro-pumps) disposed on one or more substrates (e.g., wafer(s)). In some examples, the micro-pumps include a silicon-based material. In some examples, the micro-pumps include a metal (e.g., steel) based material. In some examples, the pump 120 is non-mechanical (e.g., without moving parts).

The passive valve 121 may be defined by one or more materials within the pump 120. The passive valve 121 may allow (or prevent) fluid to enter (or exit) a fluid chamber of the pump 120. In some examples, the passive valve 121 includes a helical valve. The passive valve 121 may transition between an open position (in which fluid flows through the passive valve 121) and a closed position (in which fluid is prevented from flowing through the passive valve 121) according to the presence or absence of an external force. For example, an external force (e.g., fluid) is used to open and close the passive valve 121, where the passive valve 121 may not actuate unless there is a flow of fluid around and through a pump chamber of the pump 120.

The passive valve 121 may assist with maintaining pressure in the inflatable member 104. In some examples, a pump 120 may include a single passive valve 121. In some examples, the pump 120 may include multiple passive valves 121 such as two passive valves or more than two passive valves 121. The passive valve 121 may not be directly controlled by the controller 114, but rather based on the pressure between the inflatable member 104 and the fluid reservoir 102. The passive valve 121 may transition between an open position (in which fluid is permitted to flow through the passive valve 121) and a closed position (in which fluid is prevented from flowing through the passive valve 121. In some examples, the passive valve 121 transitions to the closed position in response to positive pressure between the inflatable member 104 and the fluid reservoir 102. In some examples, the passive valve 121 transitions to the open position in response to negative pressure between the inflatable member 104 and the fluid reservoir 102.

In some examples, the flow modifier 145 is disposed in a fluid passageway 117 connected to the pump 120. The flow modifier 145 is configured to restrict the flow of fluid entering the pump 120. In some examples, the flow modifier 145 is disposed in a fluid passageway 117 between the fluid reservoir 102 and the pump 120. In some examples, the electronic pump assembly 106 includes multiple flow modifiers 145, e.g., a first flow modifier 145 disposed in the fluid passageway 117 between the fluid reservoir 102 and the pump 120 (e.g., connected to an input of the pump 120) and a second flow modifier 145 disposed in a fluid passageway 119 between the pump 120 and an inflatable member 104 (e.g., connected to an output of the pump 120).

In some examples, referring to FIG. 1B, the flow modifier 145 includes a tubular member 156 and a filter member 158 disposed within a cavity of the tubular member 156. In some examples, the filter member 158 includes a disc member. In some examples, the filter member 158 includes a cylindrical member. In some examples, the filter member 158 is disposed in a central region within the cavity of the tubular member 156. The tubular member 156 may be disposed within the fluid passageway 117 and/or the fluid passageway 119.

The filter member 158 includes a first end portion 162 and a second end portion 164. In some examples, the first end portion 162 includes a flat surface. In some examples, the first end portion 162 includes a curved portion. In some examples, the first end portion 162 includes a conical portion. In some examples, the second end portion 164 includes a conical portion. The filter member 158 defines a plurality of holes 160. The holes 160 may extend between the first end portion 162 and the second end portion 164. The holes 160 may be through-holes that extend entirely through the filter member 158. In some examples, the holes 160 include linear holes (e.g., straight holes). In some examples, the holes 160 include conical holes. In some examples, the holes 160 includes y-shaped holes. In some examples, the holes 160 include hexagonal holes or honeycomb holes. In some examples, the filter member 158 defines the holes 160 in a gyroid lattice (e.g., a structure with channels in multiple directions). In some examples, the sum of the area of the holes 160 is substantially the same as the area of the filter member 158. In some examples, the sum of the area of the holes 160 is substantially the same as the area of the tubular member 156.

In some examples, the flow modifier 145 is defined by one or more materials of the passive valve 121. In some examples, the material(s) of the passive valve 121 may have different material properties or dimensions to enable a stiffer valve. For example, the material(s) of the passive valve 121 may be constructed from a material that includes a higher stiffness than other materials of the pump 120 (e.g., the material of a diaphragm actuator). In some examples, the material(s) of the passive valve 121 may have a thickness greater than the thickness of other materials of the pump 120 (e.g., the material of a diaphragm actuator). In some examples, the passive valve(s) 121 include helical valves and the diameter(s) of the helical valves may be different (e.g., different from each other) and/or non-symmetrical to change the flow rate.

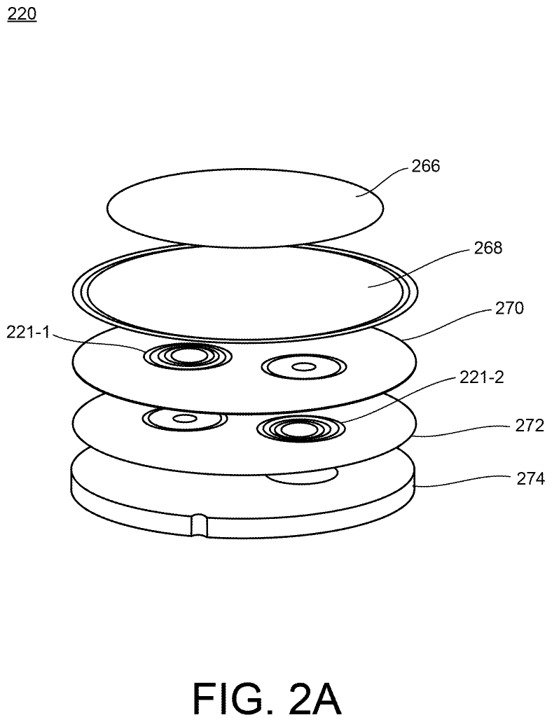

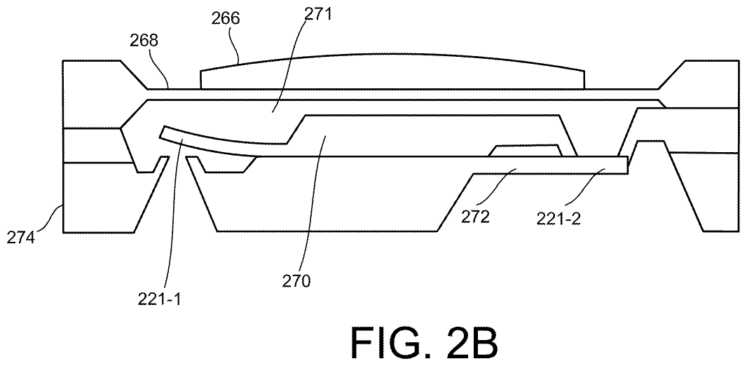

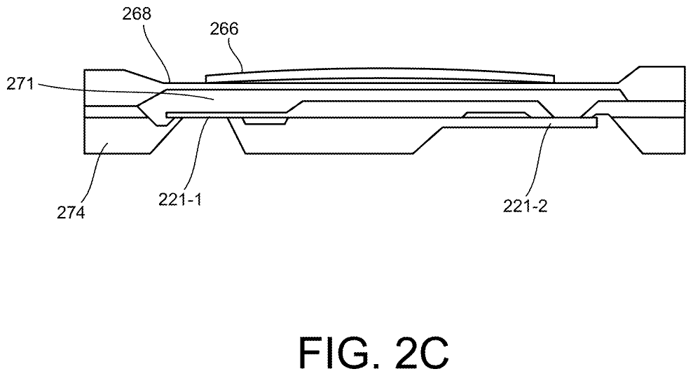

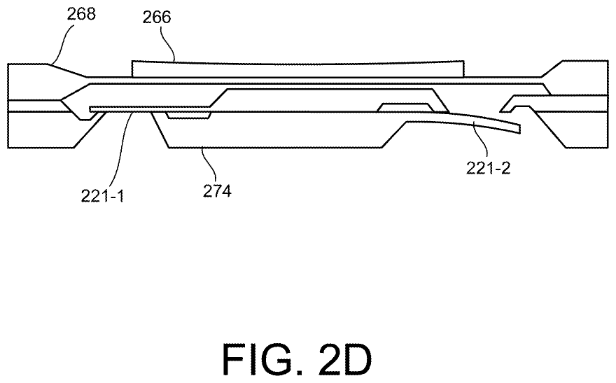

FIGS. 2A through 2D illustrate an example of a pump 220. The pump 220 may be a piezoelectric diaphragm pump. In some examples, the pump 220 is a cymbal-type piezoelectric pump. In some examples, the pump 220 may include any type of piezoelectric actuator that could be used to actuate a diaphragm (e.g., temperature-based, pneumatic pressure-based, and/or electromagnetic, etc.). The pump 220 includes a piezo element 266 configured to receive a voltage from the controller 114, a diaphragm actuator 268 coupled to the piezo element 266, and a base plate 274 defining an inlet port and an outlet port. In some examples, the piezo element 266 has a disc shape that has a diameter smaller than a diameter of the diaphragm actuator 268. In some examples, the diaphragm actuator 268 has a disc shape. In some examples, the diaphragm actuator 268 includes a metal-based material. In some examples, the diaphragm actuator 268 includes a titanium diaphragm. In some examples, the base plate 274 has a disc shape. The base plate 274 defines an inlet port that extends through the thickness of the base plate 274. The base plate 274 defines an outlet port that extends through the thickness of the base plate 274. The thickness of the base plate 274 may be larger than the thickness of the piezo element 266 and the diaphragm actuator 268. In some examples, the base plate 274 has a diameter that is substantially the same (e.g., within 2 mm) as the diaphragm actuator 268.

The pump 220 includes a layer of material 270 defining a passive valve 221-1 (e.g., an inlet check valve) and a layer of material 272 defining a passive valve 221-2 (e.g., an outlet check valve). The layer of material 270 and the layer of material 272 may be titanium. The layer of material 270 and the layer of material 272 may have a disc shape. The layer of material 270 and the layer of material 272 may have a diameter that is substantially the same (e.g., within 2 mm) as the base plate 274 and/or the diaphragm actuator 268. The layer of material 270 may define one or more helical slots that form the passive valve 221-1. The passive valve 221-1 may be disposed on top of the inlet port of the base plate 274. The layer of material 272 may define one or more helical slots that form the passive valve 221-1. The passive valve 221-2 may be disposed on top of the outlet port of the base plate 274.

FIG. 2B illustrates the pump 220 in a supply mode, where fluid is pumped into a fluid chamber 271 when the piezo element 266 causes the diaphragm actuator 268 to bend upwards. During the supply mode when fluid is entering the fluid chamber 271, the passive valve 221-1 (e.g., the inlet check valve) is open and the passive valve 221-2 (e.g., the outlet check valve) is closed. FIG. 2C illustrates the pump 220 at the end of the supply mode when both the passive valve 221-1 and the passive valve 221-2 are closed (e.g., the fluid chamber 271 is full). FIG. 2D illustrates the pump 220 in a pump mode in which fluid is expelled from the fluid chamber 271 when the piezo element 266 causes the diaphragm actuator 268 to bend downwards. For example, during the pump mode, the passive valve 221-1 is closed and the passive valve 221-2 is open to allow the fluid to flow out of the fluid chamber 271.

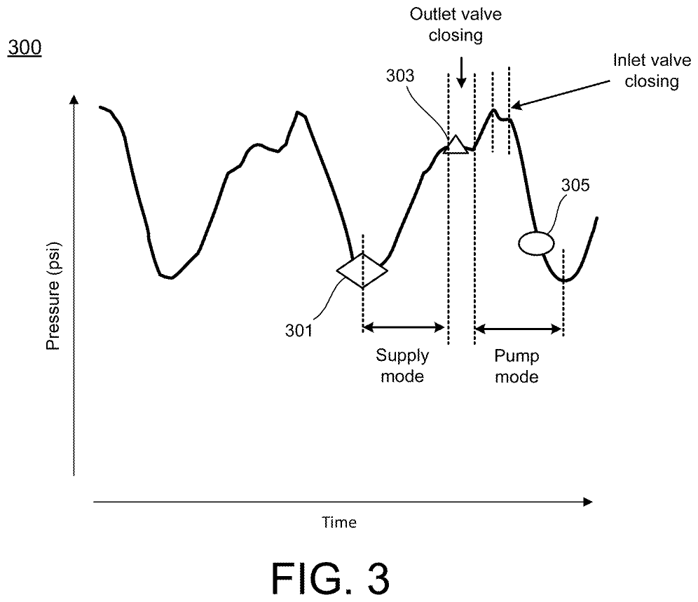

FIG. 3 illustrates a pressure graph 300 depicting the pressure of a pump during the pumping cycle when an electronic pump assembly does not include a flow modifier. For example, the supply mode starts at element 301 in which the pump has the pump configuration of FIG. 2B and ends at element 303 in which the pump has the pump configuration of FIG. 2C. The pump mode begins after the supply mode, where, during the pump mode at element 305, the pump has the pump configuration of FIG. 2D. In the supply mode, the diaphragm actuator is displacing upwards, drawing fluid into the pump. As shown in FIG. 3, there is a downward dip in the pressure graph and this is the outlet valve (e.g., passive valve 221-1) closing and is marked by a decrease followed by a sharp increase in the slope of the pressure graph. A maximum pressure is reached, and, at this stage, the piezo element and diaphragm actuator start to drive down to supply and expel the fluid from the pump. The downward slope is slight until the inlet valve (e.g., passive valve 221-1) is closed, allowing the force of the piezo element to act to expel the fluid from the pump leading to a sharper slope on the pressure graph. However, as shown in FIG. 3, the inefficiency in the pump comes from the time taken to close both valves during the stroke.

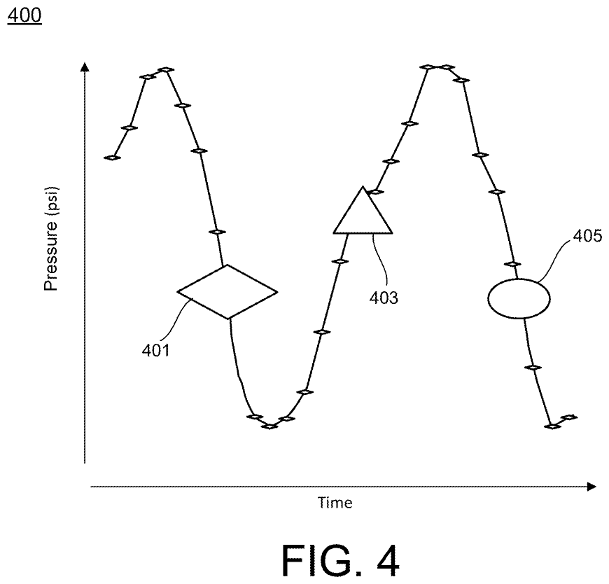

FIG. 4 illustrates a pressure graph 400 depicting the pressure of a pump during the pumping cycle when an electronic pump assembly includes a flow modifier. Element 401 depicts the pump having the pump configuration of FIG. 2B, element 403 depicts the pump having the pump configuration of FIG. 2C, and element 405 depicts the pump having the pump configuration of FIG. 2D. As shown in FIG. 4, the erratic parts of the pressure graph 300 of FIG. 3 have smooth out, thereby increasing the efficiency of the pump.

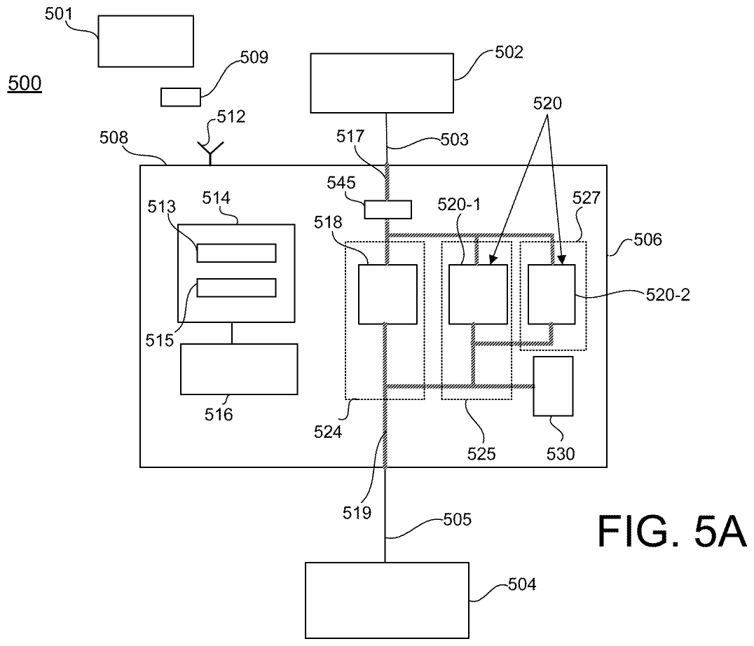

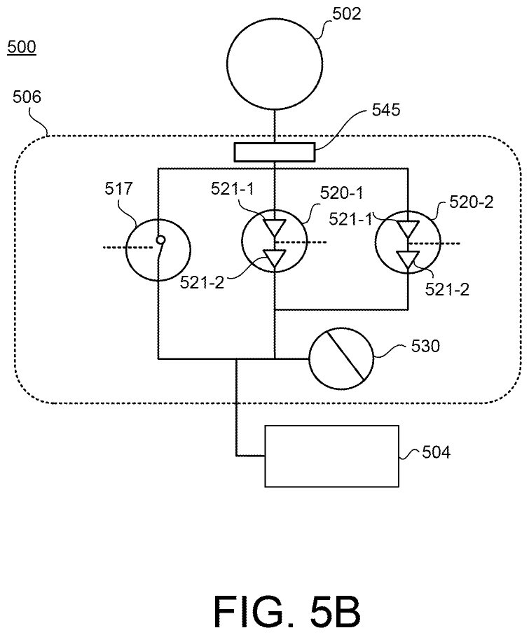

FIGS. 5A and 5B illustrate an implantable device 500 with an electronic pump assembly 108 according to an aspect. The implantable device 500 may be an example of the device 100 of FIGS. 1A and 1B and may include any of the details discussed with reference to those figures. Also, the implantable device 500 includes one or more pumps 520. The pump(s) 520 may be an example of the pump 120 of FIGS. 1A and 1B and/or the pump 220 of FIGS. 2A through 2D and may include any of the details discussed with reference to those figures.

The implantable device 500 may include a fluid reservoir 502, an inflatable member 504, and an electronic pump assembly 506 configured to transfer fluid between the fluid reservoir 502 and the inflatable member 504. In some examples, the implantable device 500 is an artificial urinary sphincter device. In some examples, the implantable device 500 is an inflatable penile prosthesis. However, the implantable device 500 may include any type of medical device that transfers fluid between components of the implantable device 500.

In some examples, the inflatable member 504 is an inflatable cuff member configured to be implemented around a urethra of a patient. In some examples, the inflatable member 504 is a penile inflation member (e.g., one or more inflatable cylinders) that may be implanted into the corpus cavernosum of the user. In some examples, the fluid reservoir 502 may be implanted in the abdomen or pelvic cavity of the user (e.g., the fluid reservoir 502 may be implanted in the lower portion of the user's abdominal cavity or the upper portion of the user's pelvic cavity). In some examples, at least a portion of the electronic pump assembly 506 may be implemented in the patient's body.

The fluid reservoir 502 may include a container having an internal chamber configured to hold or house fluid that is used to inflate the inflatable member 504. The volumetric capacity of the fluid reservoir 502 may vary depending on the size of the implantable device 500. In some examples, the volumetric capacity of the fluid reservoir 502 may be 3 to 550 cubic centimeters. In some examples, the fluid reservoir 502 is constructed from the same material as the inflatable member 504. In other examples, the fluid reservoir 502 is constructed from a different material than the inflatable member 504. In some examples, the fluid reservoir 502 contains a larger volume of fluid than the inflatable member 504.

The implantable device 500 may include a first conduit connector 503 and a second conduit connector 505. Each of the first conduit connector 503 and the second conduit connector 505 may define a lumen configured to transfer the fluid to and from the pump assembly 506. The first conduit connector 503 may be coupled to the electronic pump assembly 506 and the fluid reservoir 502 such that fluid can be transferred between the electronic pump assembly 506 and the fluid reservoir 502 via the first conduit connector 503. For example, the first conduit connector 503 may define a first lumen configured to transfer fluid between the electronic pump assembly 506 and the fluid reservoir 502. The first conduit connector 503 may include a single or multiple tube members for transferring the fluid between the electronic pump assembly 506 and the fluid reservoir 502.

The second conduit connector 505 may be coupled to the pump assembly 506 and the inflatable member 504 such that fluid can be transferred between the electronic

The electronic pump assembly 506 and the inflatable member 504 via the second conduit connector 505. For example, the second conduit connector 505 may define a second lumen configured to transfer fluid between the electronic pump assembly 506 and the inflatable member 504. The second conduit connector 505 may include a single or multiple tube members for transferring the fluid between the electronic pump assembly 506 and the inflatable member 504. In some examples, the first conduit connector 503 and the second conduit connector 505 may include a silicone rubber material. In some examples, the electronic pump assembly 506 may be directly connected to the fluid reservoir 502.assembly 506 may automatically transfer fluid between the fluid reservoir 502 and the inflatable member 504 without the user manually operating a pump (e.g., squeezing and releasing a pump bulb). The electronic pump assembly 506 that can monitor control and regulate a pressure within an inflatable member 504. The electronic pump assembly 506 may include a controller 514, one or more active valves 518, one or more pumps 520, and a pressure sensor 530 (or multiple pressure sensors). The controller 514 may control the pump(s) 520 and the active valve(s) 518 to move fluid between the inflatable member 504 and the fluid reservoir 502 to transition the inflatable member 504 between an inflation state and a deflation state. The pressure sensor 530 may monitor the pressure of the inflatable member 504. The controller 514 may receive pressure readings from the pressure sensor 530 and control the pump(s) 520 and the active(s) valves 518 to maintain and/or adjust the pressure of the inflatable member 504. The controller 514 may send control signals to the pump(s) 520 and the active valve(s) 518 to inflate or deflate the inflatable member 504. In some examples, the control of the inflation state and the deflation state is based on wireless signals 509 received from an external device 501 that is operated by the patient (and the detected pressure of the inflatable member 504 from the pressure sensor 530). For example, the patient may use the external device 501 to place the inflatable member 504 in an inflation or deflation state, which causes the external device 501 to send a wireless signal 509 to the controller 514.

The electronic pump assembly 506 includes a filter modifier 545 disposed within a fluid passageway 517 between the pump(s) 520 and the fluid reservoir 502. The filter modifier 545 may be an example of the filter modifier 145 of FIGS. 1A and 1B and may include any of the details discussed with reference to those figures.

The electronic pump assembly 506 may include a battery 516 configured to provide power to the controller 514 and other components on the electronic pump assembly 506. In some examples, the battery 516 is a non-rechargeable battery. In some examples, the battery 516 is a rechargeable battery. In some examples, the electronic pump assembly 506 (or a portion thereof) (or the controller 514) is configured to be connected to an external charger to charge the battery 516. In some examples, the electronic pump assembly 506 may define a charging interface that is configured to connect to the external charger. In some examples, the charging interface includes a universal serial bus (USB) interface configured to receive a USB charger. In some examples, the charging technology may be electromagnetic or Piezoelectric.

The electronic pump assembly 506 may include an antenna 512 configured to wirelessly transmit (and receive) wireless signals 509 from an external device 501. The external device 501 may be any type of component that can communicate with the electronic pump assembly 506. The external device 501 may be a computer, smartphone, tablet, pendant, key fob, etc. A user may use the external device 501 to control the implantable device 500. In some examples, the user may use the external device 501 to inflate or deflate the inflatable member 504. For example, in response to the user activating an inflation cycle using the external device 501 (e.g., selecting a user control on the external device 501), the external device 501 may transmit a wireless signal 509 to the electronic pump assembly 506 to initiate the inflation cycle (received via the antenna 512), where the controller 514 may control the active valve(s) 518 and the pump(s) 520 to inflate the inflatable member 504 to a target inflation pressure. In some examples, the controller 514 may cause the active valve 518 to a closed position and cause the pump(s) to operate to move fluid from the fluid reservoir 502 to the inflatable member 504.

In some examples, in response to the user activating a deflation cycle using the external device 501 (e.g., selecting a user control on the external device 501), the external device 501 may transmit a wireless signal 509 to the electronic pump assembly 506 to initiate the deflation cycle (received via the antenna 512), where the controller 514 may control the active valve(s) 518 (and, in some examples, the pump(s) 520) to transfer fluid from the inflatable member 504 to the fluid reservoir 502. For example, the controller 514 may control the active valve 518 to move to the open position to allow fluid to transfer from the inflatable member 504 to the fluid reservoir 502. In some examples, the controller 514 may control one or more pumps 520 to further move the fluid from the inflatable member 504 to the fluid reservoir 502 during the deflation cycle. In some examples, during the deflation cycle, fluid is transferred back until the pressure in the inflatable member 504 reaches a partial inflation pressure. In some examples, the controller 514 may automatically determine to initiate a deflation cycle, which causes the controller 514 to control the active valve(s) 518 (and, in some examples, the pump(s) 520) to transfer fluid back to the fluid reservoir 502.

The controller 514 may be any type of controller configured to control operations of the pump(s) 520 and the active valve(s) 518. In some examples, the controller 514 is a microcontroller. In some examples, the controller 514 includes one or more drivers configured to drive the pump(s) 520 and the active valve(s) 518. In some examples, the driver(s) are components separate from the controller 514. The controller 514 may be communicatively coupled to the active valve(s) 518, the pump(s) 520, and the pressure sensor(s) 530. In some examples, the controller 514 is connected to the active valve(s) 518, the pump(s) 520, and the pressure sensor(s) 530 via wired data lines. The controller 514 may include a processor 513 and a memory device 515.

The processor 513 may be formed in a substrate configured to execute one or more machine executable instructions or pieces of software, firmware, or a combination thereof. The processor 513 can be semiconductor-based — that is, the processors can include semiconductor material that can perform digital logic. The memory device 515 may store information in a format that can be read and/or executed by the processor 513. The memory device 515 may store executable instructions that when executed by the processor 513 cause the processor 513 to perform certain operations discussed herein. The controller 514 may receive data via the pressure sensor(s) 530 and/or the external device 501 and control the active valve(s) 518 and/or the pump(s) 520 by transmitting control signals to the active valve(s) 518 and/or the pump(s) 520.

The memory device 515 may store control parameters that can be set or modified by the user and/or physician using the external device 501. In some examples, the control parameters may include the target inflation pressure and/or the partial inflation pressure. In some examples, the target inflation pressure is a maximum (or desired) pressure allowable in the inflatable member 504. In some examples, the partial inflation pressure is a pressure threshold that can more closely mimic the natural experience and/or personal comfort of the user. A user or physician may update the control parameters using the external device 501, which can be communicated to the controller 514 via the antenna 512 and then updated in the memory device 515.

The external device 501 may communicate with the electronic pump assembly 506 over a network. In some examples, the network includes a short-range wireless network such as near field communication (NFC), Bluetooth, or infrared communication. In some examples, the network may include the Internet (e.g., Wi-Fi) and/or other types of data networks, such as a local area network (LAN), a wide area network (WAN), a cellular network, satellite network, or other types of data networks.

In some examples, the electronic pump assembly 506 includes a single pump 520 such as a pump 520-1. The pump 520-1 may be disposed in parallel with the active valve 518. In some examples, the electronic pump assembly 506 includes multiple pumps 520. For example, the pumps 520 include pump 520-1 and pump 520-2. In some examples, the pump 520-1 is disposed in a fluid passageway 525 that is used to fill the inflatable member 504 (e.g., during the inflation cycle). In some examples, the pump 520-2 is disposed in a fluid passageway 527 that is used to fill the inflatable member 504 (e.g., during the inflation cycle). In some examples, the pump 520-2 is disposed in parallel with the pump 520-1. The pump 520-1 may transfer fluid according to a first flow rate, and the pump 520-1 may transfer fluid according to a second flow rate. In some examples, the first flow rate is substantially the same as the second flow rate. In some examples, the first flow rate is different from the second flow rate.

In some examples, the pumps 520 may include more than two pumps 520 such as three, four, five, six, or greater than six pumps 520. For example, the pumps 520 may include a third pump in parallel with the pump 520-2, a fourth pump in parallel with the third pump, and so forth. In some examples, the pumps 520 may include one or more pumps 520 in series with one or more other pumps 520. For example, one or more pumps 520 may be in series with the pump 520-1. In some examples, one or more pumps 520 may be in series with the pump 520-2.

Each pump 520 is an electronically-controlled pump. Each pump 520 may be electronically-controlled by the controller 514. For example, each pump 520 may be connected to the controller 514 and may receive a signal to actuate a respective pump 520. A pump 520 may be unidirectional in which the pump 520 can transfer fluid from the fluid reservoir 502 to the inflatable member 504 (or from the inflatable member 504 to the fluid reservoir 502). In some examples, a pump 520 is bidirectional in which the pump 520 can transfer fluid from the fluid reservoir 502 to the inflatable member 504 and from the inflatable member 504 to the fluid reservoir 502. In some examples, the pumps 520 are either unidirectional or bidirectional. In some examples, the pumps 520 include a combination of one or more unidirectional pumps and one or more bidirectional pumps.

In some examples, the pump 520 is an electromagnetic pump that moves the fluid between the fluid reservoir 502 and the inflatable member 504 using electromagnetism. With respect to an electromagnetic pump, a magnetic fluid is set at angles to the direction the fluid moves in, and a current is passed through it.

In some examples, the pump 520 is a piezoelectric pump. In some examples, a piezoelectric pump may be a diaphragm micropump that uses actuation of a diaphragm to drive a fluid. In some examples, a piezoelectric pump may include one or more piezo pumps (e.g., piezo elements), which may be implemented by a substrate layer (e.g., a single substrate layer) of high-voltage piezo elements or may be implemented by multiple substrate layers (e.g., stacked substrate layers) of low-voltage piezo elements. In some examples, the pump 520 includes a plurality of micro-pumps (e.g., piezoelectrically-driven micro-pumps) disposed on one or more substrates (e.g., wafer(s)). In some examples, the micro-pumps include a silicon-based material. In some examples, the micro-pumps include a metal (e.g., steel) based material. In some examples, the pump 520 is non-mechanical (e.g., without moving parts).

In some examples, in the case of multiple pumps 520, each pump 520 may be a pump of the same type (e.g., all pumps 520 are electromagnetic pumps or all pumps 520 are piezoelectric pumps). In some examples, one or more pumps 520 are different from one or more other pumps 520. For example, pumps 520 may include different types of piezoelectric pumps or the pumps 520 may include different types of electromagnetic pumps. The pump 520-1 may be a piezoelectric pump having a first number of micro-pumps, and the pump 520-2 may be a piezoelectric pump having a second number of micro-pumps (where the second number is different from the first number). The pump 520-1 may be an electromagnetic pump, and the pump 520-2 may be a piezoelectric pump.

As shown in FIG. 5B, a pump 520 may include one or more passive valves such as passive valve 521-1 and passive valve 521-2. The passive valve(s) may assist with maintaining pressure in the inflatable member 504. In some examples, a pump 520 may include a single passive valve. In some examples, the pump 520 may include multiple passive valves such as two passive valves or more than two passive valves. The passive valve(s) of a respective pump 520 may not be directly controlled by the controller 514, but rather based on the pressure between the inflatable member 504 and the fluid reservoir 502. The passive valve(s) may transition between an open position (in which fluid is permitted to flow through the passive valve(s)) and a closed position (in which fluid is prevented from flowing through the passive valve(s)). In some examples, the passive valve(s) transitions to the closed position in response to positive pressure between the inflatable member 504 and the fluid reservoir 502. In some examples, the passive valve(s) transition to the open position in response to negative pressure between the inflatable member 504 and the fluid reservoir 502.

The electronic pump assembly 506 may include a single active valve 518. In some examples, the electronic pump assembly 506 includes multiple active valves 518. In some examples, one or more additional active valves 518 may be in series with a pump 520-1 and/or a pump 520-2. In some examples, an additional active valve 518 (e.g., a series active valve 518) may be disposed in a fluid passageway 517 that is connected to the fluid reservoir 502. In some examples, an additional active valve 518 (e.g., a series active valve 518) may be disposed in a fluid pathway portion 519 that is connected to the inflatable member 504. These additional active valves 518 may reduce leakage when at maximum inflation pressure or at partial inflation pressure.

The active valve 518 may be connected to the controller 514 of the electronic pump assembly 506 and may receive a signal to transition the active valve 518 between the open position and the closed position. In some examples, the active valve 518 is disposed in a fluid passageway 524 that is used to empty the inflatable member 504 (e.g., in the deflation cycle). In some examples, the active valve 518 is disposed in a fluid passageway 524 that is used to fill the inflatable member 504 (e.g., in the inflation cycle). In some examples, the active valve 518 may transition to the closed position to hold (e.g., substantially hold) the pressure in the inflatable member 504. In some examples, the active valve 518 may transition to the open position to transfer fluid back to the fluid reservoir 502, release pressure in the inflatable member 504 and/or allow a flow back to the inflatable member 504. In some examples, the active valve 518 may be used to hold (e.g., substantially hold) a partial inflation pressure.

In some examples, the electronic pump assembly 506 includes a hermetic enclosure 508 that encloses the components of the electronic pump assembly 506. A hermetic enclosure 508 may be an air-tight (or substantially air-tight) container. The hermetic enclosure 508 may include one or more metal-based materials. In some examples, the hermetic enclosure 508 is a Titanium container. In some examples, the only material in contact with the patient is Titanium. In some examples, the hermetic enclosure 508 includes one or more non-metal-based materials (e.g., ceramic). In some examples, a portion of the hermetic enclosure 508 is a metal-based material and a portion of the hermetic enclosure 508 is a non-metal-based material. In some examples, the hermetic enclosure 508 defines a feedthrough (e.g., a hermetic feedthrough, an electrical feedthrough, a feedthrough connector, etc.) to receive/transmit wireless signals from/to the external device 501. In some examples, the feedthrough includes a metal-based material and an insulator-based material (e.g., ceramic).

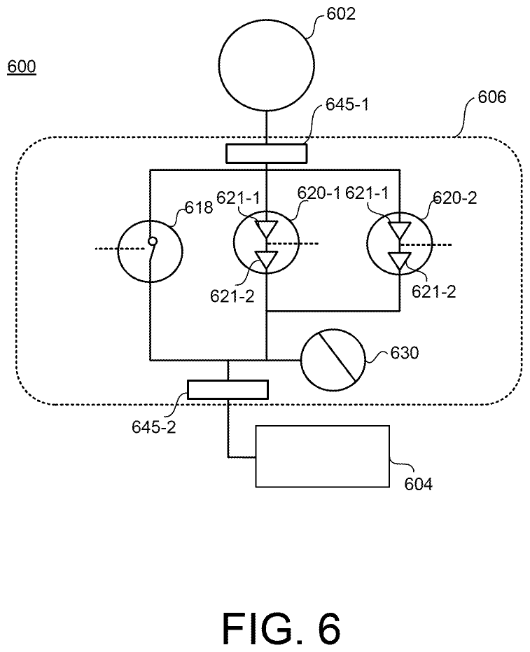

FIG. 6 illustrates an example of an implantable device 600 according to another aspect. The implantable device 600 may be an example of the device 100 of FIGS. 1A and 1B and the implantable device 500 of FIGS. 5A and 5B and may include any of the details discussed with reference to those figures. The implantable device 600 includes a fluid reservoir 602, an inflatable member 604, and an electronic pump assembly 606. The electronic pump assembly 606 includes an active valve 618, a pump 620-1, a pump 620-2, and a pressure sensor 630. The pump 620-1 includes a passive valve 621-1 and a passive valve 621-2. The pump 620-2 includes a passive valve 621-1 and a passive valve 621-2. The pump 620-1 and the pump 620-2 may be examples of the pump 220 of FIGS. 2A through 2D and may include any of the details discussed with reference to those figures.

The electronic pump assembly 606 includes a flow modifier 645-1 disposed within a fluid passageway between the fluid reservoir 602 and the pumps 620-1, 620-2. The electronic pump assembly 606 includes a flow modifier 645-2 disposed within a fluid passageway between the inflatable member 604 and the pumps 620-1, 620-2. The flow modifier 645-1 and the flow modifier 645-2 may be examples of the flow modifier 145 of FIGS. 1A and 1B and may include any of the details discussed with reference to those figures.

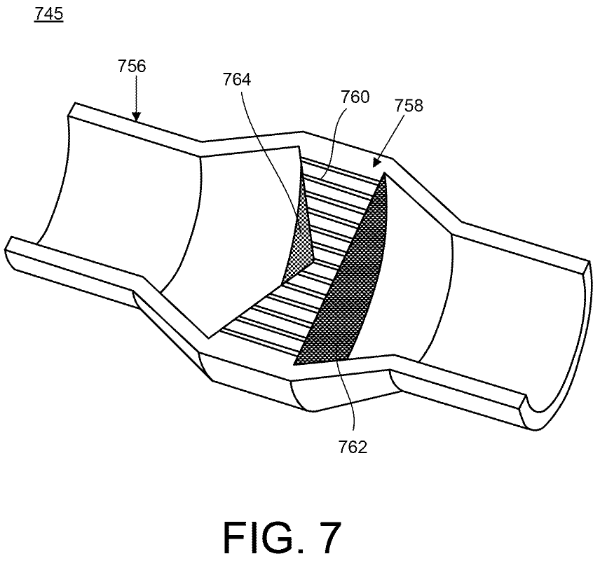

FIG. 7 illustrates an example of a flow modifier 745 configured as a filter according to an aspect. The flow modifier 745 may be an example of the flow modifier 145 of FIGS. 1A and 1B, the flow modifier 545 of FIGS. 5A and 5B, and/or the flow modifier 645 of FIG. 6 and may include any of the details discussed with reference to those figures.

The flow modifier 745 includes a tubular member 756 and a filter member 758 disposed within a cavity of the tubular member 756. In some examples, the filter member 758 includes a disc member. In some examples, the filter member 758 includes a cylindrical member. In some examples, the filter member 758 is disposed in a central region within the cavity of the tubular member 756.

The filter member 758 includes a first end portion 762 and a second end portion 764. In some examples, the first end portion 762 includes a flat surface. In some examples, the first end portion 762 includes a curved portion. In some examples, the first end portion 762 includes a conical portion. In some examples, the second end portion 764 includes a conical portion. The filter member 758 defines a plurality of holes 760. The holes 760 may extend between the first end portion 762 and the second end portion 764. The holes 760 may be through-holes that extend entirely through the filter member 758. In some examples, the holes 760 include linear holes (e.g., straight holes), as shown in FIG. 7. In some examples, the holes 760 include conical holes. In some examples, the holes 760 includes y-shaped holes. In some examples, the holes 760 include hexagonal holes or honeycomb holes. In some examples, the filter member 758 defines the holes 760 in a gyroid lattice (e.g., a structure with channels in multiple directions). In some examples, the sum of the area of the holes 760 is substantially the same as the area of the filter member 758. In some examples, the sum of the area of the holes 760 is substantially the same as the area of the tubular member 756.

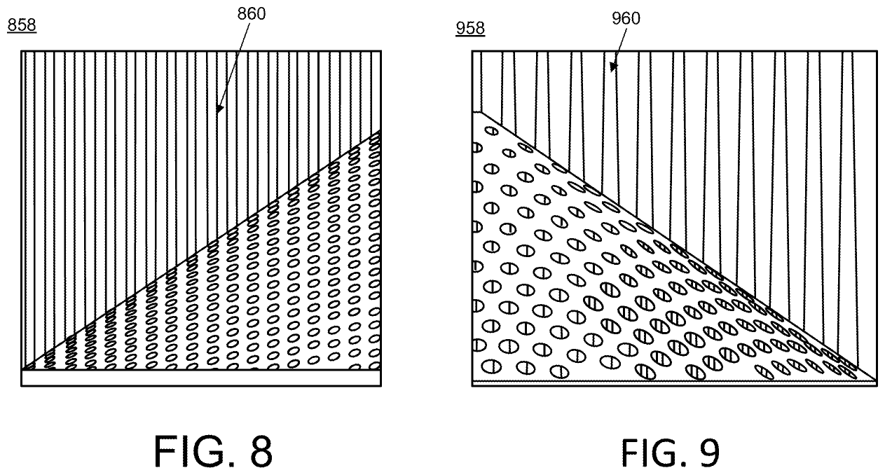

FIG. 8 illustrates a portion of a filter member 858 depicting straight or linear holes 860. FIG. 9 illustrates a portion of a filter member 958 depicting conical holes 960.

FIGS. 10A and 10B illustrate a flow modifier 1045 having a tubular member 1056 and a filter member 1058 disposed within a cavity of the tubular member 1056. The filter member 1058 includes a first end portion 1062 and a second end portion 1064. The first end portion 1062 may include a flat surface. The second end portion 1064 may include a conical portion. FIG. 10B illustrates a portion of the filter member 1058. The filter member 1058 defines a plurality of holes 1060. In some examples, the holes 1060 are linear or straight holes.

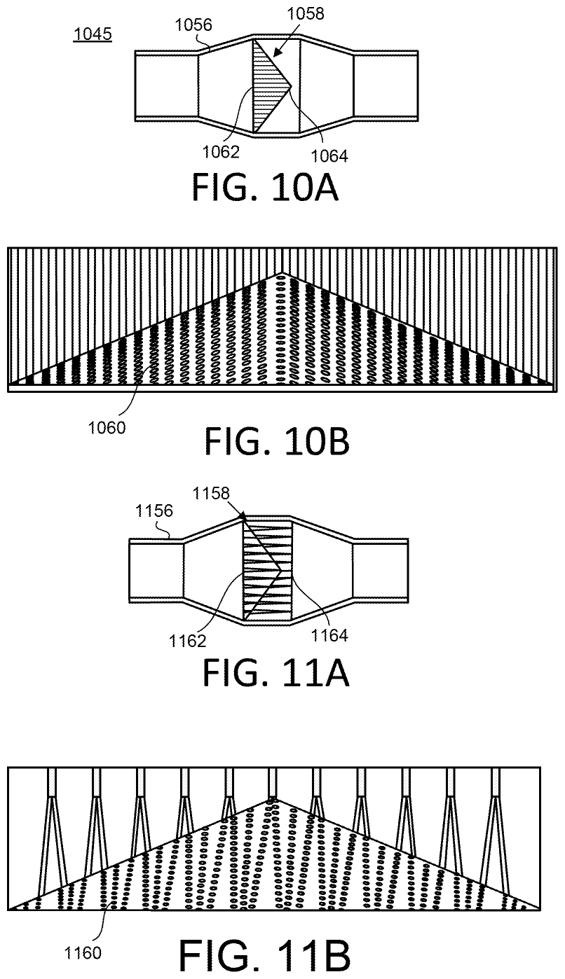

FIGS. 11A and 11B illustrate a flow modifier 1145 having a tubular member 1156 and a filter member 1158 disposed within a cavity of the tubular member 1156. The filter member 1158 includes a first end portion 1162 and a second end portion 1164. The first end portion 1162 may include a flat surface. The second end portion 1164 may include a conical portion. FIG. 11B illustrates a portion of the filter member 1158. The filter member 1158 defines a plurality of holes 1160. In some examples, the holes 1160 are y-shaped holes.

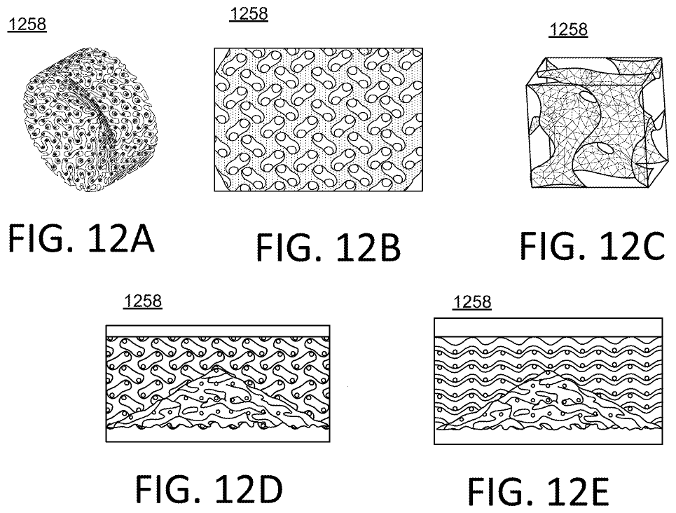

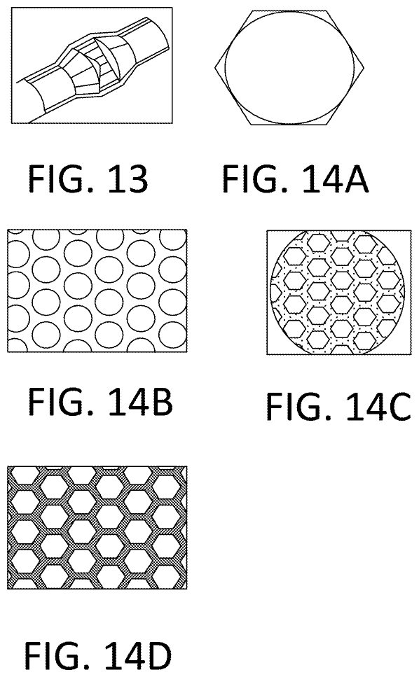

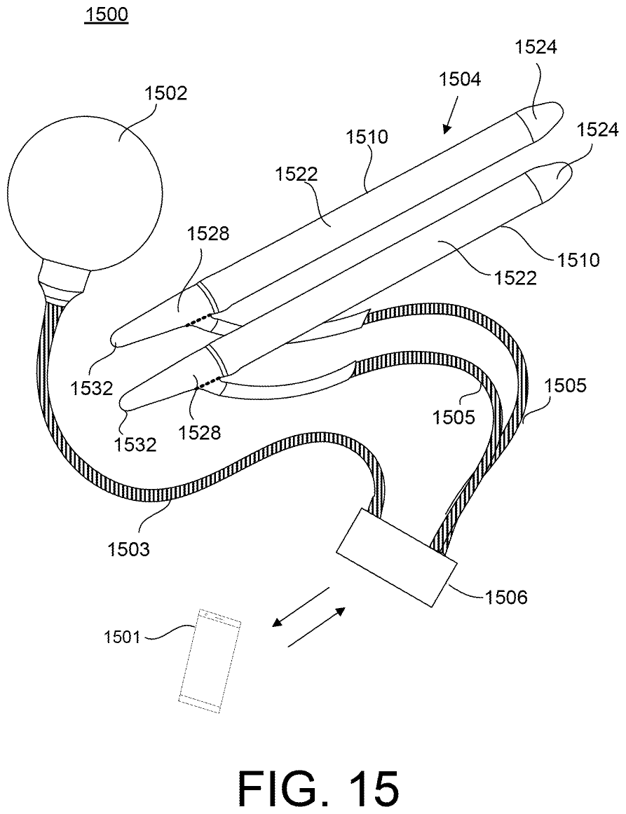

FIGS. 12A through 12E illustrate a filter member 1258 defining a plurality of holes that are arranged in a Gyroid lattice. In some examples, a Gyroid lattice is a minimal surface structure with channels in multiple directions that could trap fibers and particulate in its structure. FIG. 13 illustrates a flow modifier according to another aspect. FIGS. 14A through 14D illustrate hexagonal holes or honeycomb holes.

FIG. 15 schematically illustrates an inflatable penile prosthesis 1500 having an electronic pump assembly 1506 according to an aspect. The electronic pump assembly 1506 may include any of the features of the electronic pump assembly discussed herein. The inflatable penile prosthesis 1500 may include an inflatable member 1504 (e.g., a pair of inflatable cylinders 1510), and the inflatable cylinders 1510 are configured to be implanted in a penis. For example, one of the inflatable cylinders 1510 may be disposed on one side of the penis, and the other inflatable cylinder 1510 may be disposed on the other side of the penis. Each inflatable cylinder 1510 may include a first end portion 1524, a cavity or inflation chamber 1522, and a second end portion 1528 having a rear tip 1532.

At least a portion of the electronic pump assembly 1506 may be implanted in the patient's body. A pair of conduit connectors 1505 may attach the electronic pump assembly 1506 to the inflatable cylinders 1510 such that the electronic pump assembly 1506 is in fluid communication with the inflatable cylinders 1510. Also, the electronic pump assembly 1506 may be in fluid communication with a fluid reservoir 1502 via a conduit connector 1503. The fluid reservoir 1502 may be implanted into the user's abdomen. The inflation chamber 1522 of the inflatable cylinder 1510 may be disposed within the penis. The first end portion 1524 of the inflatable cylinder 1510 may be at least partially disposed within the crown portion of the penis. The second end portion 1528 may be implanted into the patient's pubic region PR with the rear tip 1532 proximate to the pubic bone PB.

In order to implant the inflatable cylinders 1510, the surgeon first prepares the patient. The surgeon often makes an incision in the penoscrotal region, e.g., where the base of the penis meets with the top of the scrotum. From the penoscrotal incision, the surgeon may dilate the patient's corpus cavernosum to prepare the patient to receive the inflatable cylinders 1510. The corpus cavernosum is one of two parallel columns of erectile tissue forming the dorsal part of the body of the penis, e.g., two slender columns that extend substantially the length of the penis. The surgeon will also dilate two regions of the pubic area to prepare the patient to receive the second end portion 1528. The surgeon may measure the length of the corpora cavernosum from the incision and the dilated region of the pubic area to determine an appropriate size of the inflatable cylinders 1510 to implant.

After the patient is prepared, the inflatable penile prosthesis 1500 is implanted into the patient. The tip of the first end portion 1524 of each inflatable cylinder 1510 may be attached to a suture. The other end of the suture may be attached to a needle member (e.g., Keith needle). The needle member is inserted into the incision and into the dilated corpus cavernosum. The needle member is then forced through the crown of the penis. The surgeon tugs on the suture to pull the inflatable cylinder 1510 into the corpus cavernosum. This is done for each inflatable cylinder 1510 of the pair. Once the inflation chamber 1522 is in place, the surgeon may remove the suture from the tip. The surgeon then inserts the second end portion 1528. The surgeon inserts the rear end of the inflatable cylinder 1510 into the incision and forces the second end portion 1528 toward the pubic bone PB until each inflatable cylinder 1510 is in place.

A user may use an external device 1501 to control the inflatable penile prosthesis 1500. In some examples, the user may use the external device 1501 to inflate or deflate the inflatable cylinders 1510. For example, in response to the user activating an inflation cycle using the external device 1501, the external device 1501 may transmit a wireless signal to the electronic pump assembly 1506 to initiate the inflation cycle to transfer fluid from the fluid reservoir 1502 to the inflatable cylinders 1510. In some examples, in response to the user activating a deflation cycle using the external device 1501, the external device 1501 may transmit a wireless signal to the electronic pump assembly 1506 to initiate the deflation cycle to transfer fluid from the inflatable cylinders 1510 to the fluid reservoir 1502. In some examples, during the deflation cycle, fluid is transferred back until the pressure in the inflatable cylinders 1510 reaches a partial inflation pressure.

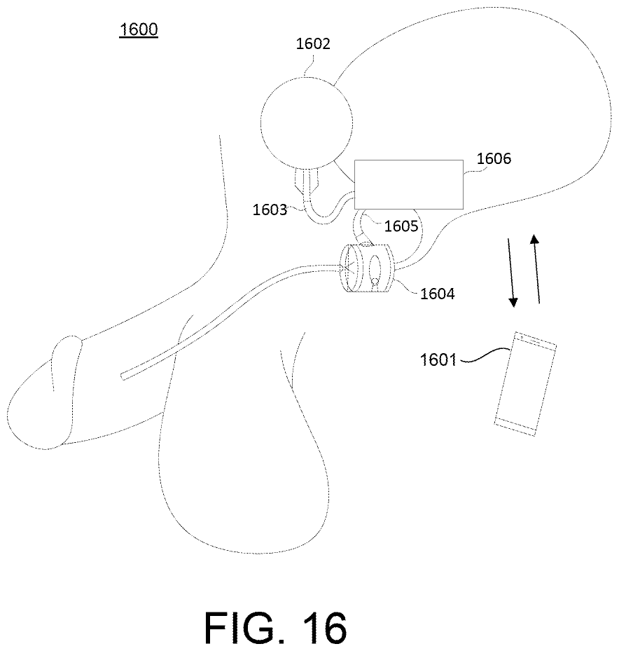

FIG. 16 illustrates a urinary control device 1600 having an electronic pump assembly 1606 according to an aspect. The electronic pump assembly 1606 may include any of the features of the electronic pump assembly discussed herein. The urinary control device 1600 includes a pump assembly 1606, a fluid reservoir 1602, and a cuff 1604.

The fluid reservoir 1602 may be a pressure-regulating inflation balloon or element. The fluid reservoir 1602 is in operative fluid communication with the cuff 1604 via one or more tube members 1603, 1605. The fluid reservoir 1602 is constructed of polymer material that is capable of elastic deformation to reduce fluid volume within the fluid reservoir 1602 and push fluid out of the fluid reservoir 1602 and into the cuff 1604. However, the material of the fluid reservoir 1602 can be biased or include a shape memory construct adapted to generally maintain the fluid reservoir 1602 in its expanded state with a relatively constant fluid volume and pressure. In some examples, this constant level of pressure exerted from the fluid reservoir 1602 to the cuff 1604 will keep the cuff 1604 at a desired inflated state when open fluid communication is provided between the fluid reservoir 1602 and the cuff 1604. This is largely due to the fact that only a small level of fluid displacement is required to inflate or deflate the cuff 1604. In some examples, the fluid reservoir 1602 is implanted into the abdominal space.

A user may use an external device 1601 to control the urinary control device 1600. In some examples, the user may use the external device 1601 to inflate or deflate the cuff 1604. For example, in response to the user activating an inflation cycle using the external device 1601, the external device 1601 may transmit a wireless signal to the electronic pump assembly 1606 to initiate the inflation cycle to transfer fluid from the fluid reservoir 1602 to the cuff 1604 (e.g., by opening an active valve where the pressure in the fluid reservoir 1602 causes the fluid to move through the active valve to the cuff 1604). In some examples, in response to the user activating a deflation cycle using the external device 1601, the external device 1601 may transmit a wireless signal to the electronic pump assembly 1606 to initiate the deflation cycle to transfer fluid from the cuff 1604 to the fluid reservoir 1602.

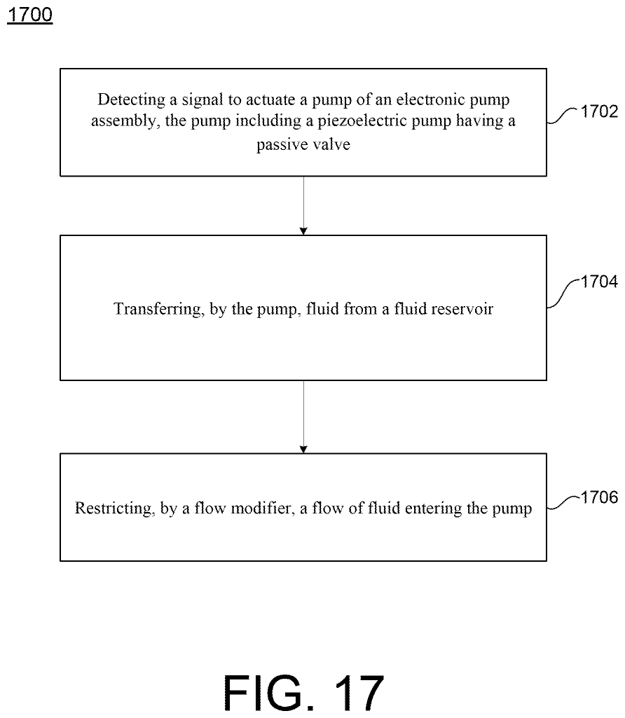

FIG. 17 illustrates a flowchart 1700 depicting example operations of operating an implantable device. The operations of the flowchart 1700 of FIG. 17 may be performed by any of the devices discussed herein.

Operation 1702 includes detecting a signal to actuate a pump of an electronic pump assembly, the pump including a piezoelectric pump having a passive valve. Operation 1704 includes transferring, by the pump, fluid from a fluid reservoir. Operation 1706 includes restricting, by a flow modifier, a flow of fluid entering the pump.

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An electronic pump assembly, the electronic pump assembly comprising:
a pump configured to move fluid between from or to a fluid reservoir of a device, the pump including a passive valve;
a controller configured to actuate the pump; and
a flow modifier configured to restrict a flow of fluid entering or within the pump.

2. The electronic pump assembly of claim 1, wherein the flow modifier includes a filter disposed within a fluid passageway connected to the pump.

3. The electronic pump assembly of claim 2, wherein the filter is a first filter connected to an input of the pump, the electronic pump assembly including a second filter connected to an output of the pump.

4. The electronic pump assembly of claim 2, wherein the filter includes a tubular member defining a cavity, the filter includes a filter component disposed within the cavity of the tubular member, the component defining a plurality of holes.

5. The electronic pump assembly of claim 4, wherein the plurality of holes include linear holes.

6. The electronic pump assembly of claim 4, wherein the plurality of holes include conical holes.

7. The electronic pump assembly of claim 4, wherein the filter component includes a cylindrical portion having a first end portion and a second end portion, the first end portion including a flat surface, the second end portion including a conical portion.

8. The electronic pump assembly of claim 1, wherein the flow modifier is defined by at least a portion of the passive valve.

9. The electronic pump assembly of claim 1, wherein the pump includes a piezo element, a diaphragm actuator, and a base plate defining an inlet port and an outlet port.

10. The electronic pump assembly of claim 9, wherein the pump includes a first layer of material defining a first passive valve and a second layer of material defining a second passive valve, the first and second layers of material being disposed between the diaphragm actuator and the base plate.

11. An implantable device comprising:
a fluid reservoir configured to hold fluid;
an inflatable member; and
an electronic pump assembly configured to transfer the fluid between the fluid reservoir and the inflatable member, the electronic pump assembly including:
a pump configured to move fluid between from or to a fluid reservoir of a device, the pump including a first passive valve and a second passive valve;
a controller configured to actuate the pump; and
a flow modifier configured to restrict a flow of fluid entering or within the pump.

12. The implantable device of claim 11, wherein the flow modifier includes a filter disposed within a fluid passageway connected to the pump.

13. The implantable device of claim 12, wherein the filter includes a tubular member defining a cavity, the filter includes a cylindrical member disposed within the cavity of the tubular member, the cylindrical member defining a plurality of holes.

14. The implantable device of claim 13, wherein the plurality of holes include circular holes, Y-shaped holes, hexagonal holes, honeycomb holes, or holes arranged in a Gyroid lattice.

15. The implantable device of claim 11, wherein the flow modifier is defined by at least a portion of the passive valve.

16. The implantable device of claim 11, wherein the pump includes a piezo element, a diaphragm actuator, and a base plate defining an inlet port and an outlet port.

17. The implantable device of claim 16, wherein the pump includes a first layer of material defining the first passive valve and a second layer of material defining the second passive valve, the first and second layers of material being disposed between the diaphragm actuator and the base plate.

18. The implantable device of claim 17, wherein at least one of the first layer of material or the second layer of material is stiffer than a material of the diaphragm actuator.

19. A method of controlling an implantable device, the method comprising;
detecting a signal to actuate a pump of an electronic pump assembly, the pump including a piezoelectric pump having a passive valve;
transferring, by the pump, fluid from a fluid reservoir; and
restricting, by a flow modifier, a flow of fluid entering the pump.

20. The method of claim 19, wherein the flow modifier is disposed in a fluid passageway between the fluid reservoir and the pump.
